(19) United States
(12) Patent Application Publication
(10) Pub. No.: US 2007/0264317 A1
(43) Pub. Date: Nov. 15, 2007

(54) Imiquimod cream formulation

(75) Inventors: Ido Yosha, Hanegev (IL); Hila Tsahor, Sde Boker (IL); Tatiana Tikhonenko, Beer Sheva (IL); Ronen Naim, Beer Sheva (IL)

Correspondence Address: PEARL COHEN ZEDEK LATZER, LLP, 1500 BROADWAY 12TH FLOOR, NEW YORK, NY, 10036, US

(73) Assignee: PERRIGO ISRAEL PHARMACEUTICALS LTD.

(21) Appl. No.: 11/433,471

(22) Filed: May 15, 2006

(51) Int. Cl. A61F 13/02; A61K 9/14; A61K 31/44

(52) U.S. Cl. 424/448; 424/489; 514/292

(57) ABSTRACT

Novel compositions containing imiquimod which are suitable for use in the treatment of skin disorders are disclosed. The compositions comprise micronized imiquimod and pharmaceutically acceptable excipients.

### FIELD OF THE INVENTION

The present invention relates to novel compositions containing imiquimod {1-(2-methylpropyl)-1H-imidazo[4,5-c]-quinolin-4-amine}, which are suitable for use in the treatment of topical disorders, including actinic keratosis, superficial basal cell carcinoma, external genital warts and more.

### BACKGROUND OF THE INVENTION

Condylomatum acuminatum is a contagious projecting warty growth on the external genitals or at the anus, consisting of fibrous overgrowths covered by thickened epithelium showing koilocytosis, due to sexual contact with infection by human pimiquimodlloma virus; it is usually benign, although malignant change has been reported, associated with particular types of the virus.

The traditional treatment options for this growing health problem are limited, painful, and tissue-destructive (surgical or caustic agents). Some years ago, the FDA approved Aldara, a topical cream containing 5% by weight of imiquimod as an active ingredient, and 25% isostearic acid as a solubilizing agent, for topical use in treating Condylomatum acuminatum. Aldara is marketed by 3M Health Care Ltd. Aldara is applied directly to the wart area 3 times per week, prior to sleeping hours, and left on the skin for 6-8 hours.

U.S. Pat. Nos. 5,238,944 and 5,736,553 to Wick, et al. and U.S. Pat. No. 6,245,776 to Skwierezynski, et al., which are attached hereto in their entirety, teach topical imiquimod compositions, in which between 0.5% to 9% of imiquimod is dissolved in either isostearic acid, oleic acid or a mixture thereof in a total amount of 3-45%, and uses thereof.

In a number of cases, topical products contain chemicals which may result idiosincrasy, namely, an abnormal susceptibility to a drug, protein, or other agent which is peculiar to the individual. The existence of single product containing imiqiomod in the market, which includes large amount of isostearic acid may be problematic to a specific population due to this phenomenon. In general, it can be assumed that reduction in the concentration of a fatty acid in a pharmaceutical formulation may be beneficial from the above described point of view. It is further noted that isostearic acid is supplied by a limited number of producers.

Formulations which included micronized imiquimod, were found ineffective at inducing pharmacological activity (Chollet et. al., Pharmaceutical Development and Technology 4(1) 35-43, 1999).

Thus, as described herein above, there is an unmet need for a formulation which contains smaller amount of fatty acids that may cause idiosyncracy, and specifically does not contain isostearic acid which is supplied by a limited number of producers.

### SUMMARY OF THE INVENTION

The primary object of the invention is to provide topical pharmaceutical compositions of imiquimod essentially free of isostearic acid for the treatment of topical disorders, including actinic keratosis, superficial basal cell carcinoma, external genital warts and more.

In an embodiment of the invention, the topical pharmaceutical compositions of imiquimod are stable.

In an embodiment of the invention the composition of the invention includes an amount of fatty acids, which is less than 25% w/w.

In an embodiment of the invention, the composition is essentially free of isostearic acid.

In some embodiments of the invention there is provided a pharmaceutical composition for topical administration comprising: a) a therapeutically effective amount of imiquimod, wherein at least part of the imiquimod is micronized; and b) a pharmaceutically acceptable excipient.

The concentration of the imiquimod in the composition of the invention may range from about 1.0% to about 7.0% w/w.

In some embodiments of the invention, the composition further comprises vehicles such as oleic acid with a combination of oleyl alcohol or stearic acid or both oleyl alcohol and stearic acid.

Other objects and advantages of the present invention will become apparent from the following description, taken in connection with the accompanying examples, wherein by way of illustration and example, an embodiment of the present invention is disclosed.

### DETAILED DESCRIPTION OF THE EMBODIMENTS OF THE INVENTION

In the following detailed description, numerous specific details are set forth in order to provide a thorough understanding of the invention. However, it will be understood by those of ordinary skill in the art that the present invention may be practiced without these specific details. In other instances, well-known methods, procedures, formulation and compositions have not been described in detail so as not to obscure the present invention.

The primary object of the invention is to provide topical pharmaceutical compositions of imiquimod essentially free of isostearic acid for the treatment of skin disorders including actinic keratosis, superficial basal cell carcinoma, external genital warts and more. The inventors were searching for a formulation which will be stable, will cause little or 110 irritation and will have the required smoothness and viscosity.

By the term “essentially free of isostearic acid” it is meant that the compositions of the invention will not contain significant amounts of isostearic acid.

By the term “significant amounts” it is meant less than 0.05% w/w of isostearic acid in the total composition.

An extensive research was conducted in order to identify a vehicle for imiquimod which is different than isostearic acid. As is presented in the Examples, formulations that were prepared with different agents like coconut oil, castor oil, glycerin, glyceryl monostearate and sorbitol demonstrated stability and the required physical properties. In addition, the compositions required micronization of the imiquimod in order to achieve good penetration of the active ingredient.

In an embodiment of the invention, there is provided a topical composition in which at least part of the imiquimod is micronized.

By the term “at least part of the imiquimod is micronized” it is meant that at least part (2-100%) of the imiquimod added to the formulation is milled or grinded or otherwise brought to particles, which are about 10 microns or less.

In an embodiment of the invention, the imiquimod is micronized to a particle size in the range of 1 to 7 microns.

In another embodiment of the invention, the imiquimod is micronized to a particle size which is less than about 3 microns.

In another embodiment of the invention, 50% of the imiquimod is micronized to a size of less than 3 microns.

In another embodiment of the invention, 70% of the imiquimod is micronized to a size of less than 3 microns.

In another embodiment of the invention, 90% of the imiquimod is micronized to a size of less than 3 microns.

It has been further surprisingly found that a dermal penetration of imiquimod in a therapeutic amount can be achieved when formulated in a composition comprising oleic acid in combination with stearic acid or oleyl alcohol or in combination with stearic acid and oleyl alcohol.

In accordance with an embodiment of the invention, there are disclosed stable topical compositions comprising imiquimod in a vehicle other than isostearic acid for treating various skin disorders, including actinic keratosis, superficial basal cell carcinoma, external genital warts and more.

In another embodiment of the invention, there are disclosed stable topical compositions for treating skin disorders such as described hereinabove, comprising micronized imiquimod in oleic acid and oleyl alcohol.

In another embodiment of the invention, there are disclosed stable topical compositions for treating skin disorders such as described hereinabove, comprising micronized imiquimod in oleic acid and stearic acid.

In another embodiment of the invention, there are disclosed stable topical compositions for treating skin disorders such as described hereinabove, comprising micornized imiquimod in oleic acid, oleyl alcohol and stearic acid.

The concentration of imiquimod in the composition ranges in an embodiment of the invention may range from about 1% to about 10% by weight.

In another embodiment of the invention, the concentration of the imiquimod is from about 1% to about 7% by weight.

In an embodiment of the invention the concentration of the imiquimod is about 5% by weight.

The formulations of the present invention comprise in an embodiment of the invention from about 1% to about 15.0% w/w of oleic acid. In another embodiment, the formulation comprises from about 5% to about 10.0% w/w. In another embodiment of the invention, the formulation comprises about 7.4% w/w.

In an embodiment of the invention, the formulation comprises about 2% to about 20.0% w/w of oleyl alcohol. In another embodiment, the formulation comprises from about 7% to about 15.0% w/w oleyl alcohol. In another embodiment of the invention, the formulation comprises about 10% w/w oleyl alcohol.

In an embodiment of the invention, the formulation comprises about 0.5% to about 8.0% w/w of stearic acid. In another embodiment, the formulation comprises from about 2% to about 4.0% w/w stearic acid. In another embodiment of the invention, the formulation comprises about 3% w/w stearic acid.

In an embodiment of the invention, the composition may comprise imiquimod at about 5.0% w/w; oleic acid at about 7.4% w/w; stearic acid at about 3.0% w/w; and a pharmaceutically acceptable excipient. At least part of the imiquimod of the invention may be micronized. In another embodiment, at least part of the imiquimod may be micronized to a particle size of less than 5 microns.

In an embodiment of the invention the composition may comprise imiquimod at about 5.0% w/w; oleic acid at about 7.4% w/w; oleyl alcohol at about 10.0% w/w; and a pharmaceutically acceptable excipient. At least part of the imiquimod of the invention may be micronized. In another embodiment, at least part of the imiquimod may be micronized to a particle size of less than 5 microns.

In an embodiment of the invention the composition may comprise imiquimod at about 5.0% w/w; oleic acid at about 7.4% w/w; oleyl alcohol at about 10.0% w/w; stearic acid at about 3.0% w/w; and a pharmaceutically acceptable excipient. At least part of the imiquimod of the invention may be micronized. In another embodiment, at least part of the imiquimod may be micronized to a particle size of less than 5 microns.

In an embodiment of the invention, the topical pharmaceutical compositions of imiquimod of the invention are chemically and physically stable.

In an embodiment of the invention, the composition may be applied for the topical treatment of clinical typical nonhyperkeratotic, nonhypertrophic actinic keratoses on the face or scalp in immunocompetent adults. Also, it may be applied for the treatment of external genital and perianal warts/condyloma acuminata in individuals 12 years old and above.

The topical compositions of this invention may further contain pharmaceutically acceptable excipients as is well known to anyone skilled in the art of pharmacology. Such excipients can be, without limitations from about 1.0% to about 5.0% w/w of one or more thickening agents and/or gelling agents, such as, but not limited to, cellulosic ethers, gums, acrylic acid polymers, Carbopol, etc. as well as from about 0.1% to about 3.0% w/w of inorganic thickeners/gelling agents, from about 1.0% to about 10.0% w/w of one or more humectants, such as glycerin for hydrating the skin and emollients, like white petrolatum for softening and smoothing the skin, from about 0.01% to about 3.0% w/w of one or more pH stabilizing agents, from about 0.01% to about 3.0% w/w of one or more preservatives such as propyl paraben, methyl paraben, benzyl alcohol, from about 1.0% to about 30.0% w/w of one or more vehicles.

The compositions of the invention are formulated in an embodiment of the invention as creams. However, other topical forms, such as, for example, lotions, gels, emulsions, patches, shampoo, solutions, foams, pastes, mousses, aerosols, ointments, etc., may be formulated.

According to another aspect of the invention, there are provided topically applied stable pharmaceutical combinations and formulations comprising, together with pharmaceutical excipients suitable for topical application, imiquimod, and a high concentration of linoleic acid. A high linoleic acid concentration is required for dissolving the imiquimod in topical formulations. The formulations of the invention comprise from about 15.0% to about 45.0% of linoleic acid, more preferably from about 20.0% to about 30.0%, and even more preferably about 25.0%.

The above methods may employ the use of the formulation or combination of the invention by applying the formulation or combination several times a week (for example 3 times a week) for a certain period of time, for example, 16 weeks, so as to clear the lesion.

### EXAMPLES

The following examples further describe and demonstrate embodiments within the scope of the subject invention. In said examples to follow, all percentages are given by weights. The examples are given solely for the purpose of illustration and are not to be constructed as limitations of the subject invention, as many variations thereof are possible without departing from the spirit and scope of the invention.

### Example 1

Imiquimod was formulated into a cream suitable for topical application as shown in Table 1:

TABLE 1

| | Concentration in the Formulation |
|---|---|
| Component | of the invention (% w/w) |
| Imiquimod | 5.0 |
| Oleic acid | 7.4 |
| Stearic acid | 3.0 |
| Cetyl alcohol | 2.2 |
| Stearyl alcohol | 3.1 |
| White petrolatum | 3.0 |
| Polysorbate 60 | 3.4 |
| Sorbitan monostearate | 0.6 |
| Glycerin | 2.0 |
| Xanthan gum | 0.5 |
| Benzyl alcohol | 2.0 |
| Methylparaben | 0.2 |
| Propylparaben | 0.02 |
| Purified Water | 67.58 |

### Example 2

Additional cream formulation of imiquimod suitable for topical application was prepared as shown in Table 2.

TABLE 2

| | Concentration in the Formulation |
|---|---|
| Component | of the invention (% w/w) |
| Imiquimod | 5.0 |
| Oleic acid | 7.4 |
| Oleyl alcohol | 10.0 |
| Cetyl alcohol | 2.2 |
| Stearyl alcohol | 3.1 |
| White petrolatum | 3.0 |
| Polysorbate 60 | 3.4 |
| Sorbitan monostearate | 0.6 |
| Glycerin | 2.0 |
| Xanthan gum | 0.5 |
| Benzyl alcohol | 2.0 |
| Methylparaben | 0.2 |
| Propylparaben | 0.02 |
| Purified Water | 60.58 |

### Example 3

Additional cream formulation of imiquimod suitable for topical application was prepared as shown in Table 3.

TABLE 3

| | Concentration in the Formulation |
|---|---|
| Component | of the invention (% w/w) |
| Imiquimod | 5.0 |
| Oleic acid | 7.4 |
| Oleyl alcohol | 10.0 |
| Stearic acid | 3.0 |
| Cetyl alcohol | 2.2 |
| Stearyl alcohol | 3.1 |
| White petrolatum | 3.0 |
| Polysorbate 60 | 3.4 |
| Sorbitan monostearate | 0.6 |
| Glycerin | 2.0 |
| Xanthan gum | 0.5 |
| Benzyl alcohol | 2.0 |
| Methylparaben | 0.2 |
| Propylparaben | 0.02 |
| Purified Water | 57.58 |

### Example 4

Additional cream formulation of imiquimod for topical application was prepared as shown in Table 4.

TABLE 4

| | Concentration in the Formulation |
|---|---|
| Component | of the invention (% w/w) |
| Imiquimod | 5.0 |
| Linoleic acid | 25.0 |
| Cetyl alcohol | 2.2 |
| Stearyl alcohol | 3.1 |
| White petrolatum | 3.0 |
| Polysorbate 60 | 3.4 |
| Sorbitan monostearate | 0.6 |
| Glycerin | 2.0 |
| Xanthan gum | 0.5 |
| Benzyl alcohol | 2.0 |
| Methyl paraben | 0.2 |
| Propyl paraben | 0.02 |
| Purified Water | 52.98 |

### Example 5

Additional cream formulation of imiquimod for topical application was prepared as shown in Table 5.

TABLE 5

| | Concentration in the Formulation |
|---|---|
| Component | of the invention (% w/w) |
| Imiquimod | 5.0 |
| Sorbitol 70% | 30.0 |
| Polysorbate 60 | 3.4 |
| Oleic acid | 7.4 |
| Stearyl Alcohol | 3.1 |
| White Petrolatum | 3.0 |
| Cetyl Alcohol | 2.2 |
| Benzyl Alcohol | 2.0 |
| Glycerin | 2.0 |
| Sorbitan Monostearate | 0.6 |
| Xanthan Gum | 0.5 |
| Methylparaben | 0.2 |
| Citric acid monohydrate | 0.13 |
| Propylparaben | 0.02 |
| Purified Water | 40.45 |

### Example 6

Additional cream formulation of imiquimod for topical application was prepared as shown in Table 6.

TABLE 6

| | Concentration in the Formulation |
|---|---|
| Component | of the invention (% w/w) |
| Imiquimod | 5.0 |
| Oleic acid | 7.4 |
| Castor oil | 15.0 |
| Lactic acid | 5.0 |
| Urea | 5.0 |
| Polysorbate 60 | 3.4 |
| Stearyl Alcohol | 3.1 |
| White Petrolatum | 3.0 |
| Cetyl Alcohol | 2.2 |
| Benzyl Alcohol | 2.0 |
| Glycerin | 2.0 |
| Sorbitan Monostearate | 0.6 |
| Xanthan Gum | 0.5 |
| Methylparaben | 0.2 |
| Ammonium hydroxide solution | 2.0 |
| Propylparaben | 0.02 |
| Purified Water | 43.58 |

What is claimed is:

1. A pharmaceutical composition for topical administration comprising:
a therapeutically effective amount of imiquimod, wherein at least part of the imiquimod is micronized; and
a pharmaceutically acceptable excipient.

2. The composition of claim 1, wherein the concentration of said imiquimod ranges from about 1.0% to about 7.0% w/w.

3. The composition of claim 1, wherein the concentration of said imiquimod is about 5.0% w/w.

4. The composition of claim 1, wherein said composition comprises oleic acid with a combination of oleyl alcohol or stearic acid or both oleyl alcohol and stearic acid.

5. The composition of claim 4, wherein the concentration of said oleic acid ranges from about 1.0% to about 15.0% w/w.

6. The composition of claim 4, wherein the concentration of said oleic acid ranges from about 5.0% to about 10.0% w/w.

7. The composition of claim 4, wherein the concentration of said oleic acid is about 7.4% w/w.

8. The composition of claim 4, wherein the concentration of said oleyl alcohol ranges from about 5.0% to about 15.0% w/w.

9. The composition of claim 4, wherein the concentration of said oleyl alcohol is about 10%.

10. The composition of claim 4, wherein the concentration of said stearic acid ranges from 0.5% to about 8% w/w.

11. The composition of claim 4, wherein the concentration of said stearic acid is about 3%.

12. The composition of claim 1, wherein said composition is essentially free of isostearic acid.

13. The compositions of claim 1, being in a form of a cream, a gel, an ointment, a lotion, a foam, a suppository, an emulsion, a paste, a mixture, a spray, a solution, a mousse, patches, or an aerosol.

14. The composition of claim 1 for treating skin disorders including actinic keratosis, superficial basal cell carcinoma, external genital warts.

15. A pharmaceutical composition according to claim 1 suitable for topical administration, comprising:

| | |
|---|---|
| imiquimod | 5.0% w/w; |
| oleic acid | 7.4% w/w; |
| stearic acid | 3.0% w/w; and |

a pharmaceutically acceptable excipient,
wherein at least part of the imiquimod is micronized.

15. The composition of claim 14, wherein the imiquimod is micronized to a particle size of less than 5 microns.

16. A pharmaceutical composition according to claim 1 suitable for topical administration, comprising:

| | |
|---|---|
| imiquimod | 5.0% w/w; |
| oleic acid | 7.4% w/w; |
| oleyl alcohol | 10% w/w; and |

a pharmaceutically acceptable excipient,
wherein at least part of the imiquimod is micronized.

17. The composition of claim 16, wherein the imiquimod is micronized to a particle size of less than 5 microns.

18. A pharmaceutical composition according to claim 1 suitable for topical administration, comprising:

| | |
|---|---|
| imiquimod | 5.0% w/w; |
| oleic acid | 7.4% w/w; |
| oleyl alcohol | 10% w/w; |
| stearic acid | 3%; w/w; and |

a pharmaceutically acceptable excipient,
wherein at least part of the imiquimod is micronized.

19. The composition of claim 18S wherein the imiquimod is micronized to a particle size of less than 5 microns.
